(19) United States
(12) Patent Application Publication
(10) Pub. No.: US 2010/0056440 A1
(43) Pub. Date: Mar. 4, 2010

(54) COMPOSITIONS AND METHODS FOR ADMINISTERING GDNF LIGAND FAMILY PROTEINS

(75) Inventors: Anthony Rossomando, South Grafton, MA (US); R. Blake Pepinsky, Arlington, MA (US)

Correspondence Address: FISH & RICHARDSON, P.O. BOX 1022, MINNEAPOLIS, MN, 55440-1022, US

(73) Assignee: Biogen Idec MA Inc., Cambridge, MA (US)

(21) Appl. No.: 12/281,077

(22) PCT Filed: Feb. 27, 2007
(86) PCT No.: PCT/US07/05366

§ 371 Date: Oct. 13, 2009

Related U.S. Application Data
(60) Provisional application No. 60/778,509, filed on Mar. 1, 2006.

(51) Int. Cl. A61K 38/18

(52) U.S. Cl. 514/12

(57) ABSTRACT

Disclosed are methods of increasing serum exposure of an administered glial cell line-derived neurotrophic factor (GDNF) ligand family protein by administering to a subject via systemic delivery (i) a GDNF ligand family protein, and (ii) an amount of heparin or heparan sulphate that increases serum exposure of the administered GDNF ligand family protein in the subject.

### TECHNICAL FIELD

The invention relates to protein chemistry, molecular biology, and neurobiology.

### BACKGROUND

Glial cell line-derived neurotrophic factor (GDNF) was initially identified as a critical factor for the survivability of dopaminergic neurons in the midbrain. The pattern of the seven cysteine residues within its amino acid sequence was consistent with that of transforming growth factor-beta (TGF-beta), indicating that GDNF (and related proteins) may be considered a subclass of this “superfamily” (Lin et al., 1993, Science, 260:1130). The characterization of GDNF was followed by the identification of the related growth factors Neurturin (Kotzbauer et al., 1996, Nature, 384:467), Persephin (Milbrandt et al., Neuron, 20:245), and Neublastin (also known as Artemin and Enovin) (Baloh et al., 1998, Neuron, 21:1291; Masure et al., 1999, Eur J Biochem, 266:892), which together comprise the GDNF ligand family of neurotrophic factors.

Studies with GDNF knock-out mice suggested a major role of GDNF is in the development of the enteric nervous system and regulation of renal organogenesis (Moore et al., 1996, Nature, 382:76). Neurturin was first characterized in studies aimed at examination of recombinant growth factors expressed in transfected Chinese hamster ovary (CHO) cells when it was found that one of these factors enhanced the survival of sympathetic neurons cultured from neonatal mouse superior cervical ganglia even in the presence of antiserum to NGF (Kotzbauer et al., supra). Expression cloning studies led to the characterization of Persephin which, like GDNF, promoted survival of cultured motor neurons and of midbrain dopaminergic neurons and promoted regeneration of axotomized motor neurons in neonatal rats (Milbrandt et al., supra). The most recently discovered GDNF ligand family member is Neublastin, which promotes the outgrowth and survival of neurons of the peripheral and central nervous system (Baudet et al., 2000, Development, 127:4335; Masure et al., supra; Rosenblad et al., 2000, Mol. Cell. Neurosci., 15(2):199).

All of the GDNF ligand family members act through ternary complex receptor systems containing the RET receptor tyrosine kinase as common signaling component (Baloh et al., 1998, Neuron, 21:1291; Mason, et al., 2000, Pharm Acta Helv, 74:261; Masure et al., 2000, J Biol Chem, 275:39427). Specificity is conferred by binding of the ligands to a unique GDNF family receptor alpha (GFR alpha). The GFR alpha 1 to GFR alpha 4 receptors are glycosyl-phosphatidyl inositol (GPI) anchored proteins that, when bound to the preferred GDNF ligand, activate RET. GDNF binds preferentially to GFR alpha 1, Neurturin to GFR alpha 2, Neublastin to GFR alpha 3, and Persephin to GFR alpha 4.

### SUMMARY

The invention is based, at least in part, on the discovery that co-administration of heparin with a systemically delivered GDNF ligand family protein (Neublastin) increases serum exposure of the administered protein.

Disclosed are methods of increasing serum exposure of an administered GDNF ligand family protein by administering to a subject via systemic delivery a pharmaceutical composition containing (i) a GDNF ligand family protein, and (ii) an amount of heparin or heparan sulphate that increases serum exposure of the administered GDNF ligand family protein in the subject.

As used herein, a “GDNF ligand family protein” refers to a Neublastin polypeptide, a GDNF polypeptide, a Neurturin polypeptide, or a Persephin polypeptide.

As used herein, “an amount of heparin or heparan sulphate that increases serum exposure of the administered GDNF ligand family protein” refers to an amount that results in serum levels of the protein following administration that exceed serum levels that result when the GDNF ligand family protein is administered, via the same route of administration, in the absence of heparin or heparan sulphate.

“Systemic delivery” refers to a route of administration that results in the administered protein traveling through the bloodstream and reaching cells throughout the body. Systemic delivery does not encompass localized means of delivery such as intracerebral delivery, intraventricular delivery, or intracerebroventricular delivery.

Also disclosed are methods of treating a nervous system disorder by administering to a subject that has a nervous system disorder, via systemic delivery, an effective amount of a pharmaceutical composition containing (i) a GDNF ligand family protein, and (ii) an amount of heparin or heparan sulphate that increases serum exposure of the administered GDNF ligand family protein in the subject. The nervous system disorder can be, for example, neuropathic pain or loss of pain sensitivity associated with a neuropathy. Additional examples of nervous system disorders that can be treated according to the methods are detailed herein.

In some embodiments of the methods described herein, the systemic delivery is intravenous administration. In some embodiments, the systemic delivery is subcutaneous administration.

In some embodiments of the methods described herein, the GDNF ligand family protein is a Neublastin polypeptide. The Neublastin polypeptide can, for example, contain an amino acid sequence that is at least 80% identical to amino acids 15-113 of SEQ ID NO:1, wherein the polypeptide, when dimerized, binds to a complex containing GFRalpha3 and RET. In some embodiments, the amino acid sequence is at least 90%, 95%, or 98% identical to amino acids 15-113 of SEQ ID NO:1. In some embodiments, the amino acid sequence is at least 90%, 95%, or 98% identical to SEQ ID NO:1. In some embodiments, the Neublastin polypeptide contains or consists of amino acids 15-113 of SEQ ID NO:1, amino acids 10-113 of SEQ ID NO:1, or the amino acid sequence of SEQ ID NO:1.

In some embodiments of the methods described herein, the GDNF ligand family protein is a GDNF polypeptide. The GDNF polypeptide can, for example, contain an amino acid sequence that is at least 80% identical to SEQ ID NO:2, wherein the polypeptide, when dimerized, binds to a complex containing GFRalpha1 and RET. In some embodiments, the amino acid sequence is at least 90%, 95%, or 98% identical to SEQ ID NO:2. In some embodiments, the GDNF polypeptide contains or consists of the amino acid sequence of SEQ ID NO:2.

In some embodiments of the methods described herein, the GDNF ligand family protein is a Neurturin polypeptide. The Neurturin polypeptide can, for example, contain an amino acid sequence that is at least 80% identical to SEQ ID NO:3, wherein the polypeptide, when dimerized, binds to a complex containing GFRalpha2 and RET. In some embodiments, the amino acid sequence is at least 90%, 95%, or 98% identical to SEQ ID NO:3. In some embodiments, the Neurturin polypeptide contains or consists of the amino acid sequence of SEQ ID NO:3.

In some embodiments of the methods described herein, the GDNF ligand family protein is a Persephin polypeptide. The Persephin polypeptide can, for example, contain an amino acid sequence that is at least 80% identical to SEQ ID NO:4, wherein the polypeptide, when dimerized, binds to a complex containing GFRalpha4 and RET. In some embodiments, the amino acid sequence is at least 90%, 95%, or 98% identical to SEQ ID NO:4. In some embodiments, the Persephin polypeptide contains or consists of the amino acid sequence of SEQ ID NO:4.

In some embodiments of the methods described herein, the GDNF ligand family protein is not conjugated to a polymer (e.g., a polyalkylene glycol such as polyethylene glycol). For example, the Neublastin polypeptide can be a non-polymer-conjugated Neublastin polypeptide.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is an alignment of wild type human (SEQ ID NO:5), mouse (SEQ ID NO:6), and rat (SEQ ID NO:7) pre pro Neublastin polypeptides. The left and right vertical lines indicate, respectively, the start of the 113 amino acid and 104 amino acid forms. The RRXR heparin binding motif is boxed.

FIG. 2 is an alignment of wild type human (SEQ ID NO:8), mouse (SEQ ID NO:9), and rat (SEQ ID NO:10) pre pro GDNF polypeptides. The amino acid residue at the start of the mature form of the protein is bolded and underlined.

FIG. 3 is an alignment of wild type human (SEQ ID NO:11), mouse (SEQ ID NO:12), and rat (SEQ ID NO:13) pre pro Neurturin polypeptides. The amino acid residue at the start of the mature form of the protein is bolded and underlined.

FIG. 4 is an alignment of wild type human (SEQ ID NO:14), mouse (SEQ ID NO:15), and rat (SEQ ID NO:16) pre pro Persephin polypeptides. The amino acid residue at the start of the mature form of the protein is bolded and underlined.

### DETAILED DESCRIPTION

The GDNF ligand family of proteins contains the following four members: Neublastin, GDNF, Neurturin, and Persephin. The present invention provides methods for increasing serum exposure of a systemically delivered GDNF ligand family protein (or a biologically active variant thereof) by co-administration of heparin or heparan sulphate with the protein. As disclosed in the accompanying example, co-administration of heparin with Neublastin was found to increase the area under the curve and enhance the half life of the systemically administered protein.

### Neublastin Polypeptides

Mature wild type human Neublastin is 113 amino acids in length and has the following amino acid sequence: AGGPGSRARAAGARGCRLRSQLVPVRALGLG HRSDELVRFRFCSGSCRRARSPHDLSLASLLGAGALRPPPGSRPVSQPCCRPTR YEAVSFMDVNSTWRTVDRLSATACGCLG (SEQ ID NO:1). Polypeptides having the amino acid sequence of SEQ ID NO:1 or biologically active variants thereof can be used in the methods described herein. A variant Neublastin polypeptide can contain one or more additions, substitutions, and/or deletions, as detailed in the following sections. Wild-type Neublastin polypeptides and biologically active variants thereof are collectively referred to herein as “Neublastin polypeptides.”

A variant Neublastin polypeptide can vary in length from the corresponding wild-type polypeptide. Although the mature human Neublastin polypeptide (SEQ ID NO:1) consists of the carboxy terminal 113 amino acids of pre pro Neublastin (SEQ ID NO:5), not all of the 113 amino acids are required to achieve useful Neublastin biological activity. Amino terminal truncation is permissible. Thus, a variant Neublastin polypeptide can contain, for example, the carboxy terminal 99, 100, 101, 102, 103, 104, 105, 106, 107, 108, 109, 110, 111, 112, or 113 amino acids of SEQ ID NO:1 (i.e., its length can be 99, 100, 101, 102, 103, 104, 105, 106, 107, 108, 109, 110, 111, 112, or 113 amino acids).

A variant Neublastin polypeptide can also vary in sequence from the corresponding wild-type polypeptide. In particular, certain amino acid substitutions can be introduced into the Neublastin sequence without appreciable loss of a Neublastin biological activity. In exemplary embodiments, a variant Neublastin polypeptide (i) contains one or more amino acid substitutions, and (ii) is at least 70%, 80%, 85%, 90%, 95%, 98% or 99% identical to SEQ ID NO:1 (or 70%, 80%, 85%, 90%, 95%, 98% or 99% identical to amino acids 15-113 of SEQ ID NO:1). A variant Neublastin polypeptide differing in sequence from SEQ ID NO:1 (or differing in sequence from amino acids 15-113 of SEQ ID NO:1) may include one or more amino acid substitutions (conservative or non-conservative), one or more deletions, and/or one or more insertions.

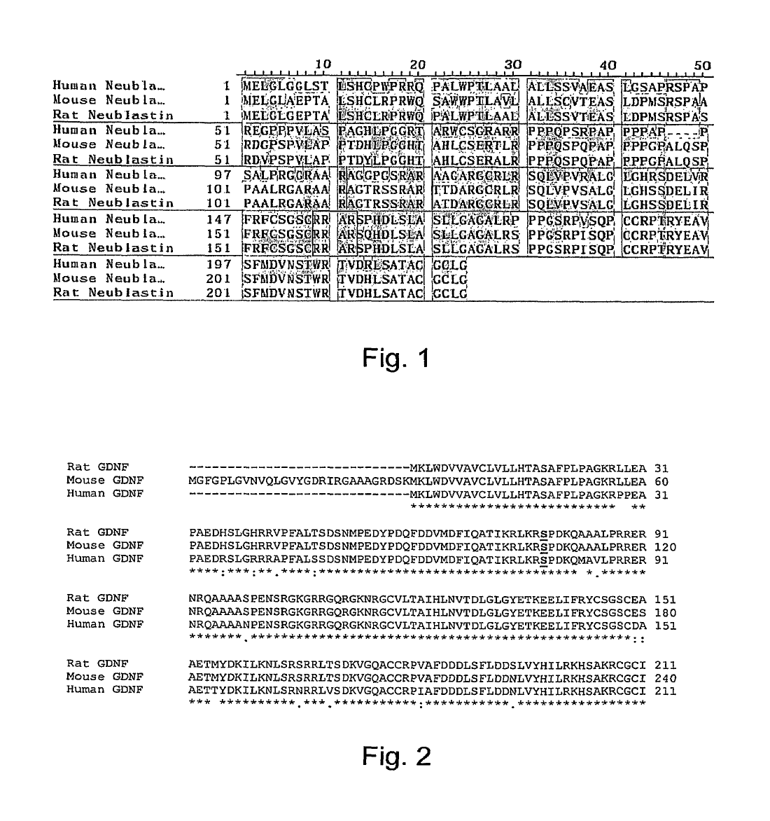

FIG. 1 is an alignment of the wild type human, mouse, and rat pre pro Neublastin polypeptides. The vertical lines in FIG. 1 indicate the start of the mature 113 amino acid form (left vertical line) and 104 amino acid form (right vertical line) of Neublastin. The RRXR heparin binding motif is boxed. This alignment of naturally occurring, bioactive forms of Neublastin indicates specific exemplary residues (i.e., those that are not conserved among the human, mouse, and rat forms) that can be substituted without eliminating bioactivity.

Percent identity between amino acid sequences can be determined using the BLAST 2.0 program. Sequence comparison can be performed using an ungapped alignment and using the default parameters (Blossom 62 matrix, gap existence cost of 11, per residue gap cost of 1, and a lambda ratio of 0.85). The mathematical algorithm used in BLAST programs is described in Altschul et al., 1997, Nucleic Acids Research 25:3389-3402.

A conservative substitution is the substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution.

Non-conservative substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, Ile, Phe or Trp) is substituted for, or by, one having a smaller side chain (e.g., Ala, Ser) or no side chain (e.g., Gly).

A biologically active variant Neublastin polypeptide, when dimerized, binds to a ternary complex containing GFRalpha3 and RET. Any method for detecting binding to this complex can be used to evaluate the biological activity a variant Neublastin polypeptide. Exemplary assays for detecting the ternary complex-binding ability of a variant Neublastin polypeptide are described in WO00/01815 (the content of which is incorporated herein by reference).

A variant Neublastin polypeptide can also be assessed to evaluate its ability to trigger the Neublastin signaling cascade. For example, the Kinase Receptor Activation (KIRA) assay can be used to assess the ability of a variant Neublastin polypeptide to induce RET autophosphorylation (See also, Sadick et al., 1996, Anal. Biochem., 235(2):207).

### GDNF Polypeptides

Mature wild type human GDNF is 134 amino acids in length and has the following amino acid sequence: SPDKQMAVLPRRERNRQAAAANPENSRGKGR RGQRGKNRGCVLTAIHLNVTDLGLGYETKEELIFRYCSGSCDAAETTYDKILK NLSRNRRLVSDKVGQACCRPIAFDDDLSFLDDNLVYHILRKHSAKRCGCI (SEQ ID NO:2). Polypeptides having the amino acid sequence of SEQ ID NO:2 or biologically active variants thereof can be used in the methods described herein. A variant GDNF polypeptide can contain one or more additions, substitutions, and/or deletions, as detailed in the following sections. Wild-type GDNF polypeptides and biologically active variants thereof are collectively referred to herein as “GDNF polypeptides.”

A variant GDNF polypeptide can vary in length from the corresponding wild-type polypeptide. Although the mature human GDNF polypeptide (SEQ ID NO:2) consists of the carboxy terminal 134 amino acids of pre pro GDNF (SEQ ID NO:8), not all of the 134 amino acids are required to achieve useful GDNF biological activity (e.g., amino terminal truncation is permissible).

A variant GDNF polypeptide can also vary in sequence from the corresponding wild-type polypeptide. In particular, certain amino acid substitutions can be introduced into the GDNF sequence without appreciable loss of a GDNF biological activity. In exemplary embodiments, a variant GDNF polypeptide (i) contains one or more amino acid substitutions, and (ii) is at least 70%, 80%, 85%, 90%, 95%, 98% or 99% identical to SEQ ID NO:2. A variant GDNF polypeptide differing in sequence from SEQ ID NO:2 may include one or more amino acid substitutions (conservative or non-conservative), one or more deletions, and/or one or more insertions.

FIG. 2 is an alignment of the wild type human, mouse, and rat pre pro GDNF polypeptides. The amino acid residue underlined and bolded in FIG. 2 indicates the start of the mature 134 amino acid form of GDNF. This alignment of naturally occurring, bioactive forms of GDNF indicates specific exemplary residues (i.e., those that are not conserved among the human, mouse, and rat forms) that can be substituted without eliminating bioactivity.

A biologically active variant GDNF polypeptide, when dimerized, binds to a ternary complex containing GFRalpha1 and RET. Any method for detecting binding to this complex can be used to evaluate the biological activity a variant GDNF polypeptide. Exemplary assays for detecting the ternary complex-binding ability of a variant GDNF polypeptide are described in WO00/01815.

A variant GDNF polypeptide can also be assessed to evaluate its ability to trigger the GDNF signaling cascade. For example, the KIRA assay can be used to assess the ability of a variant GDNF polypeptide to induce RET autophosphorylation (See also, Sadick et al., 1996, Anal. Biochem., 235(2):207).

### Neurturin Polypeptides

Mature wild type human Neurturin is 102 amino acids in length and has the following amino acid sequence: ARLGARPCGLRELEVRVSELGLGYASDETVLF RYCAGACEAAARVYDLGLRRLRQRRRLRRERVRAQPCCRPTAYEDEVSFLD AHSRYHTVHELSARECACV (SEQ ID NO:3). Polypeptides having the amino acid sequence of SEQ ID NO:3 or biologically active variants thereof can be used in the methods described herein. A variant Neurturin polypeptide can contain one or more additions, substitutions, and/or deletions, as detailed in the following sections. Wild-type Neurturin polypeptides and biologically active variants thereof are collectively referred to herein as “Neurturin polypeptides.”

A variant Neurturin polypeptide can vary in length from the corresponding wild-type polypeptide. Although the mature human Neurturin polypeptide (SEQ ID NO:3) consists of the carboxy terminal 102 amino acids of pre pro Neurturin (SEQ ID NO:11), not all of the 102 amino acids are required to achieve useful Neurturin biological activity (e.g., amino terminal truncation is permissible).

A variant Neurturin polypeptide can also vary in sequence from the corresponding wild-type polypeptide. In particular, certain amino acid substitutions can be introduced into the Neurturin sequence without appreciable loss of a Neurturin biological activity. In exemplary embodiments, a variant Neurturin polypeptide (i) contains one or more amino acid substitutions, and (ii) is at least 70%, 80%, 85%, 90%, 95%, 98% or 99% identical to SEQ ID NO:3. A variant Neurturin polypeptide differing in sequence from SEQ ID NO:3 may include one or more amino acid substitutions (conservative or non-conservative), one or more deletions, and/or one or more insertions.

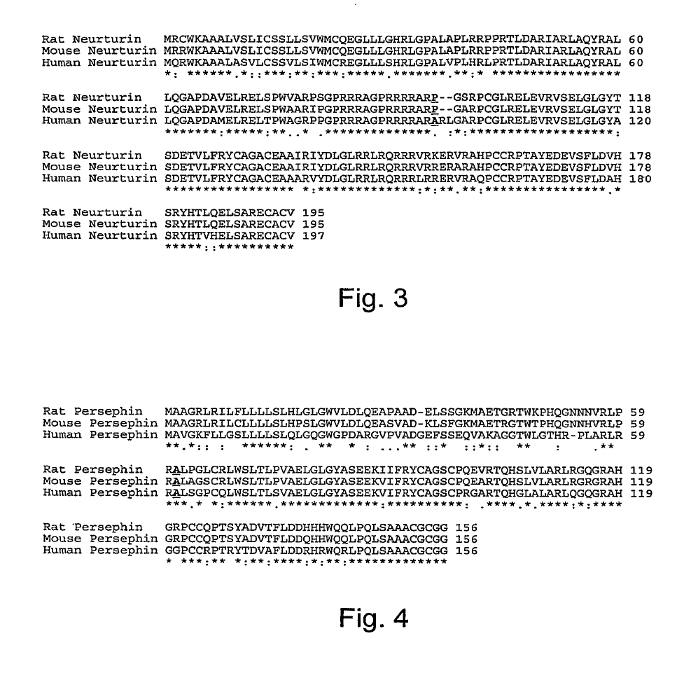

FIG. 3 is an alignment of the wild type human, mouse, and rat pre pro Neurturin polypeptides. The amino acid residue underlined and bolded in FIG. 3 indicates the start of the mature 102 amino acid form of Neurturin. This alignment of naturally occurring, bioactive forms of Neurturin indicates specific exemplary residues (i.e., those that are not conserved among the human, mouse, and rat forms) that can be substituted without eliminating bioactivity.

A biologically active variant Neurturin polypeptide, when dimerized, binds to a ternary complex containing GFRalpha2 and RET. Any method for detecting binding to this complex can be used to evaluate the biological activity a variant Neurturin polypeptide. Exemplary assays for detecting the ternary complex-binding ability of a variant Neurturin polypeptide are described in WO00/01815.

A variant Neurturin polypeptide can also be assessed to evaluate its ability to trigger the Neurturin signaling cascade. For example, the KIRA assay can be used to assess the ability of a variant Neurturin polypeptide to induce RET autophosphorylation (See also, Sadick et al., 1996, Anal. Biochem., 235(2):207).

### Persephin Polypeptides

Mature wild type human Persephin is 96 amino acids in length and has the following amino acid sequence: ALSGPCQLWSLTLSVAELGLGYASEEKVIFRY CAGSCPRGARTQHGLALARLQGQGRAHGGPCCRPTRYTDVAFLDDRHRWQ RLPQLSAAACGCGG (SEQ ID NO:4). Polypeptides having the amino acid sequence of SEQ ID NO:4 or biologically active variants thereof can be used in the methods described herein. A variant Persephin polypeptide can contain one or more additions, substitutions, and/or deletions, as detailed in the following sections. Wild-type Persephin polypeptides and biologically active variants thereof are collectively referred to herein as “Persephin polypeptides.”

A variant Persephin polypeptide can vary in length from the corresponding wild-type polypeptide. Although the mature human Persephin polypeptide (SEQ ID NO:4) consists of the carboxy terminal 96 amino acids of pre pro Persephin (SEQ ID NO:14), not all of the 96 amino acids are required to achieve useful Persephin biological activity (e.g., amino terminal truncation is permissible).

A variant Persephin polypeptide can also vary in sequence from the corresponding wild-type polypeptide. In particular, certain amino acid substitutions can be introduced into the Persephin sequence without appreciable loss of a Persephin biological activity. In exemplary embodiments, a variant Persephin polypeptide (i) contains one or more amino acid substitutions, and (ii) is at least 70%, 80%, 85%, 90%, 95%, 98% or 99% identical to SEQ ID NO:4. A variant Persephin polypeptide differing in sequence from SEQ ID NO:4 may include one or more amino acid substitutions (conservative or non-conservative), one or more deletions, and/or one or more insertions.

FIG. 4 is an alignment of the wild type human, mouse, and rat pre pro Persephin polypeptides. The amino acid residue underlined and bolded in FIG. 4 indicates the start of the mature 96 amino acid form of Persephin. This alignment of naturally occurring, bioactive forms of Persephin indicates specific exemplary residues (i.e., those that are not conserved among the human, mouse, and rat forms) that can be substituted without eliminating bioactivity.

A biologically active variant Persephin polypeptide, when dimerized, binds to a ternary complex containing GFRalpha4 and RET. Any method for detecting binding to this complex can be used to evaluate the biological activity a variant Persephin polypeptide. Exemplary assays for detecting the ternary complex-binding ability of a variant Persephin polypeptide are described in WO00/01815.

A variant Persephin polypeptide can also be assessed to evaluate its ability to trigger the Persephin signaling cascade. For example, the KIRA assay can be used to assess the ability of a variant Persephin polypeptide to induce RET autophosphorylation (See also, Sadick et al., 1996, Anal. Biochem., 235(2):207).

### Preparation of GDNF Ligand Family Proteins

A GDNF ligand family protein (e.g., a Neublastin polypeptide, a GDNF polypeptide, a Neurturin polypeptide, or a Persephin polypeptide described herein) can optionally contain heterologous amino acid sequences in addition to a GDNF ligand family protein. “Heterologous,” as used when referring to an amino acid sequence, refers to a sequence that originates from a source foreign to the particular host cell, or, if from the same host cell, is modified from its original form. Exemplary heterologous sequences include a heterologous signal sequence (e.g., native rat albumin signal sequence, a modified rat signal sequence, or a human growth hormone signal sequence) or a sequence used for purification of a GDNF ligand family protein (e.g., a histidine tag).

GDNF ligand family proteins can be isolated using methods known in the art. Naturally occurring GDNF ligand family proteins can be isolated from cells or tissue sources using standard protein purification techniques. Alternatively, mutated GDNF ligand family proteins can be synthesized chemically using standard peptide synthesis techniques. The synthesis of short amino acid sequences is well established in the peptide art. See, e.g., Stewart, et al., Solid Phase Peptide Synthesis (2d ed., 1984).

In some embodiments, GDNF ligand family proteins are produced by recombinant DNA techniques. For example, a nucleic acid molecule encoding a GDNF ligand family protein can be inserted into a vector, e.g., an expression vector, and the nucleic acid can be introduced into a cell. Suitable cells include, e.g., mammalian cells (such as human cells or CHO cells), fungal cells, yeast cells, insect cells, and bacterial cells (e.g., E. coli). When expressed in a recombinant cell, the cell is preferably cultured under conditions allowing for expression of a GDNF ligand family protein. The GDNF ligand family protein can be recovered from a cell suspension if desired. As used herein, “recovered” means that the mutated polypeptide is removed from those components of a cell or culture medium in which it is present prior to the recovery process. The recovery process may include one or more refolding or purification steps. Buffers and methods for inducing folding of a denatured GDNF ligand family protein are described in, e.g., PCT Application Number PCT/US2005/029638.

Variant GDNF ligand family proteins can be constructed using any of several methods known in the art. One such method is site-directed mutagenesis, in which a specific nucleotide (or, if desired a small number of specific nucleotides) is changed in order to change a single amino acid (or, if desired, a small number of predetermined amino acid residues) in the encoded variant GDNF ligand family protein. Many site-directed mutagenesis kits are commercially available. One such kit is the “Transformer Site Directed Mutagenesis Kit” sold by Clontech Laboratories (Palo Alto, Calif.).

### Pharmaceutical Compositions

A GDNF ligand family protein (e.g., a Neublastin polypeptide, a GDNF polypeptide, a Neurturin polypeptide, or a Persephin polypeptide described herein) can be incorporated into a pharmaceutical composition containing a therapeutically effective amount of the GDNF ligand family protein and an amount of heparin or heparan sulphate that increases serum exposure of the administered GDNF ligand family protein in a treated subject.

Heparin and heparan sulphate are chemically related alpha beta-linked glycosaminoglycans composed of alternating sequences of glucosamine and uronic acid. The size of an individual chain can reach 100 kDa, but normally they are below 50 kDa.

The heparin or heparan sulphate used in the pharmaceutical composition can be unconjugated or conjugated to another molecular entity (e.g., the heparin can be present in the form of a proteoglycan, in which the heparin is conjugated to a protein). Such conjugation is acceptable, so long as it does not eliminate the ability of the heparin or heparan sulphate to increase serum exposure of the administered GDNF ligand family protein in a treated subject. In some embodiments, the heparin or heparan sulphate is covalently linked to the GDNF ligand family protein.

A GDNF ligand family protein and heparin or heparan sulphate can be co-administered simultaneously in a single pharmaceutical composition or can be administered separately via simultaneous or sequential administrations. If administered sequentially, either the heparin or heparan sulphate or the GDNF ligand family protein can be administered first.

In addition to a GDNF ligand family protein and heparin or heparan sulphate, a pharmaceutical composition can also contain one or more adjuvants, excipients, carriers, and/or diluents. Acceptable diluents, carriers and excipients typically do not adversely affect a recipient's homeostasis (e.g., electrolyte balance). Acceptable carriers include biocompatible, inert or bioabsorbable salts, buffering agents, oligo- or polysaccharides, polymers, viscosity-improving agents, preservatives and the like. One exemplary carrier is physiologic saline (0.15 M NaCl, pH 7.0 to 7.4). Another exemplary carrier is 50 mM sodium phosphate, 100 mM sodium chloride. Further details on techniques for formulation and administration of pharmaceutical compositions can be found in, e.g., Remington's Pharmaceutical Sciences (Maack Publishing Co., Easton, Pa.).

A pharmaceutical composition containing a GDNF ligand family protein and heparin or heparan sulphate can be administered by systemic delivery. Pharmaceutical compositions can be formulated such that they are suitable for parenteral and/or non-parenteral administration. Specific administration modalities include subcutaneous, intravenous, intramuscular, intraperitoneal, transdermal, oral, rectal, buccal, nasal, intra-articular, intra-arterial, sub-arachnoid, bronchial, lymphatic, vaginal, and intra-uterine administration.

Administration may be by periodic injections of a bolus of the pharmaceutical composition or may be made more continuous by intravenous or intraperitoneal administration from a reservoir which is external (e.g., an IV bag) or internal (e.g., a bioerodible implant). See, e.g., U.S. Pat. Nos. 4,407,957, 5,798,113, and 5,800,828, each incorporated herein by reference.

Examples of parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aeorosolizer, electroporation, and transdermal patch.

Formulations suitable for parenteral administration conveniently contain a sterile aqueous preparation of the GDNF ligand family protein and heparin or heparan sulphate, which preferably is isotonic with the blood of the recipient (e.g., physiological saline solution). Formulations may be presented in unit-dose or multi-dose form.

An exemplary formulation contains a GDNF ligand family protein described herein, heparin or heparan sulphate, and the following buffer components: sodium succinate (e.g., 10 mM); NaCl (e.g., 75 mM); and L-arginine (e.g., 100 mM).

Formulations suitable for oral administration may be presented as discrete units such as capsules, cachets, tablets, or lozenges, each containing a predetermined amount of the GDNF ligand family protein and heparin or heparan sulphate; or a suspension in an aqueous liquor or a non-aqueous liquid, such as a syrup, an elixir, an emulsion, or a draught.

Therapeutically effective amounts of a pharmaceutical composition may be administered to a subject in need thereof in a dosage regimen ascertainable by one of skill in the art. For example, a composition can be administered to the subject, e.g., systemically at a dosage from 0.01 μg/kg to 1000 μg/kg body weight of the subject, per dose. In another example, the dosage is from 1 μg/kg to 100 μg/kg body weight of the subject, per dose. In another example, the dosage is from 1 μg/kg to 30 μg/kg body weight of the subject, per dose, e.g., from 3 μg/kg to 10 μg/kg body weight of the subject, per dose.

In order to optimize therapeutic efficacy, a GDNF ligand family protein is first administered at different dosing regimens. The unit dose and regimen depend on factors that include, e.g., the species of mammal, its immune status, the body weight of the mammal. Typically, protein levels in tissue are monitored using appropriate screening assays as part of a clinical testing procedure, e.g., to determine the efficacy of a given treatment regimen.

The frequency of dosing for a GDNF ligand family protein is within the skills and clinical judgement of physicians. Typically, the administration regime is established by clinical trials which may establish optimal administration parameters. However, the practitioner may vary such administration regimes according to the subject's age, health, weight, sex and medical status. The frequency of dosing may be varied depending on whether the treatment is prophylactic or therapeutic.

### Methods of Treatment

A GDNF ligand family protein (e.g., a Neublastin polypeptide, a GDNF polypeptide, a Neurturin polypeptide, or a Persephin polypeptide described herein) is useful for modulating metabolism, growth, differentiation, or survival of a nerve or neuronal cell. In particular, a GDNF ligand family protein (with heparin or heparan sulphate) can be used to treat or alleviate a disorder or disease of a living animal, e.g., a human, which disorder or disease is responsive to the activity of a neurotrophic agent.

The GDNF ligand family proteins disclosed herein (and pharmaceutical compositions comprising same) can be used in the treatment or prevention of a nervous system disorder in a subject (such as a human), by administering to a subject in need thereof a therapeutically effective amount of a GDNF ligand family protein (with heparin or heparan sulphate) or a composition containing a GDNF ligand family protein and heparin or heparan sulphate.

The nervous system disorder can be a peripheral nervous system disorder, such as a peripheral neuropathy or a neuropathic pain syndrome, or a central nervous system disorder.

A GDNF ligand family protein (administered with heparin or heparan sulphate) is useful for treating a defect in a neuron, including without limitation lesioned neurons and traumatized neurons. Peripheral nerves that experience trauma include, but are not limited to, nerves of the medulla or of the spinal cord. GDNF ligand family proteins (administered with heparin or heparan sulphate) are useful in the treatment of neurodegenerative disease, e.g., cerebral ischemic neuronal damage; neuropathy, e.g., peripheral neuropathy, Alzheimer's disease, Huntington's disease, Parkinson's disease, amyotrophic lateral sclerosis (ALS). GDNF ligand family proteins (administered with heparin or heparan sulphate) can be used in the treatment of impaired memory, e.g., memory impairment associated with dementia.

In some embodiments, motor neuron diseases such as amyotrophic lateral sclerosis (“ALS”) and spinal muscular atrophy can be treated. In other embodiments, the GDNF ligand family proteins (administered with heparin or heparan sulphate) can be used to enhance nerve recovery following traumatic injury.

In some embodiments, the GDNF ligand family proteins and heparin or heparan sulphate (and pharmaceutical compositions comprising same) are used in the treatment of various disorders in the eye, including photoreceptor loss in the retina in patients afflicted with macular degeneration, retinitis pigmentosa, glaucoma, and similar diseases.

In some embodiments, the GDNF ligand family proteins and heparin or heparan sulphate (and pharmaceutical compositions comprising same) are used for treating neuropathic pain, for treating tactile allodynia, for reducing loss of pain sensitivity associated with neuropathy, for treating viral infections and viral-associated neuropathies, and for treating painful diabetic neuropathy. The methods are discussed in detail in the following subsections.

### 1. Treatment of Neuropathic Pain

The GDNF ligand family proteins disclosed herein (and pharmaceutical compositions comprising same) can be used in methods for treating neuropathic pain in a subject comprising administering to the subject an effective amount of a GDNF ligand family protein (with heparin or heparan sulphate) alone, or by also administering to the subject an effective amount of an analgesia-inducing compound selected from the group consisting of opioids, anti-arrhythmics, topical analgesics, local anesthetics, anticonvulsants, antidepressants, corticosteroids and non-steroidal anti-inflammatory drugs (NSAIDS). In one embodiment, the analgesia-inducing compound is an anticonvulsant. In another embodiment, the analgesia-inducing compound is gabapentin ((1-aminomethyl)cyclohexane acetic acid) or pregabalin (S-(+)-4-amino-3-(2-methylpropyl)butanoic acid).

The GDNF ligand family proteins disclosed herein (and pharmaceutical compositions comprising same) can be used in the treatment of pain associated with peripheral neuropathies. Among the peripheral neuropathies which can be treated are trauma-induced neuropathies, e.g., those caused by physical injury or disease state, physical damage to the brain, physical damage to the spinal cord, stroke associated with brain damage, and neurological disorders related to neurodegeneration.

The GDNF ligand family proteins disclosed herein and heparin or heparan sulphate (and pharmaceutical compositions comprising same) can be used in the treatment of a number of peripheral neuropathies, including: (a) trauma-induced neuropathies, (b) chemotherapy-induced neuropathies, (c) toxin-induced neuropathies (including but not limited to neuropathies induced by alcoholism, vitamin B6 intoxication, hexacarbon intoxication, amiodarone, chloramphenicol, disulfiram, isoniazide, gold, lithium, metronidazole, misonidazole, nitrofurantoin), (d) drug-induced neuropathies, including therapeutic drug-induced neuropathic pain (such as caused by anti-cancer agents, particularly anti-cancer agents selected from the group consisting of taxol, taxotere, cisplatin, nocodazole, vincristine, vindesine and vinblastine; and such as caused by anti-viral agents, particularly anti-viral agents selected from the group consisting of ddI, DDC, d4T, foscarnet, dapsone, metronidazole, and isoniazid), (e) vitamin-deficiency-induced neuropathies (including but not limited to vitamin B12 deficiency, vitamin B6 deficiency, and vitamin E deficiency), (f) idiopathic neuropathies, (g) diabetic neuropathies, (h) pathogen-induced nerve damage, (i) inflammation-induced nerve damage, (j) neurodegeneration, (k) hereditary neuropathy (including but not limited to Friedreich ataxia, familial amyloid polyneuropathy, Tangier disease, Fabry disease), (l) metabolic disorders (including but not limited to renal insufficiency and hypothyroidism), (m) infectious and viral neuropathies (including but not limited to neuropathic pain associated with leprosy, Lyme disease, neuropathic pain associated with infection by a virus, particularly a virus selected from the group consisting of a herpes virus (e.g. herpes zoster which may lead to post-herpetic neuralgia), a human immunodeficiency virus (HIV), and a papilloma virus), (n) auto-immune neuropathies (including but not limited to Guillain-Barre syndrome, chronic inflammatory de-myelinating polyneuropathy, monoclonal gammopathy of undetermined significance and polyneuropathy), (o) trigeminal neuralgia and entrapment syndromes (including but not limited to Carpel tunnel), and (p) other neuropathic pain syndromes including post-traumatic neuralgia, phantom limb pain, multiple sclerosis pain, complex regional pain syndromes (including but not limited to reflex sympathetic dystrophy, causalgia), neoplasia-associated pain, vasculitic/angiopathic neuropathy, and sciatica. Neuropathic pain may be manifested as allodynia, hyperalgesia, spontaneous pain or phantom pain.

### 2. Treatment of Tactile Allodynia

The GDNF ligand family proteins disclosed herein and heparin or heparan sulphate (and pharmaceutical compositions comprising same) can be used in the treatment of tactile allodynia in a subject. The term “tactile allodynia” typically refers to the condition in a subject where pain is evoked by stimulation of the skin (e.g. touch) that is normally innocuous.

In some embodiments, tactile allodynia is treated by administering to the subject a pharmaceutically effective amount of a GDNF ligand family protein and heparin or heparan sulphate. In a related embodiment, tactile allodynia may be treated by administering to a subject an effective amount of a GDNF ligand family protein (with heparin or heparan sulphate) alone, or by administering to the subject an effective amount of a GDNF ligand family protein, heparin or heparan sulphate, and an effective amount of an analgesia-inducing compound selected from the group consisting of opioids, anti-arrhythmics, topical analgesics, local anesthetics, anticonvulsants, antidepressants, corticosteroids and NSAIDS. In one embodiment, the analgesia-inducing compound is an anticonvulsant. In another preferred embodiment, the analgesia-inducing compound is gabapentin ((1-aminomethyl)cyclohexane acetic acid) or pregabalin (S-(+)-4-amino-3-(2-methylpropyl)butanoic acid).

In some embodiments, a GDNF ligand family protein and heparin or heparan sulphate is administered in association with a therapeutic agent, including but not limited to an anti-cancer agent or an anti-viral agent. Anti-cancer agents include, but are not limited to, taxol, taxotere, cisplatin, nocodazole, vincristine, vindesine and vinblastine. Anti-viral agents include, but are not limited to, ddI, DDC, d4T, foscarnet, dapsone, metronidazole, and isoniazid.

### 3. Treatment for Reduction of Loss of Pain Sensitivity

In another embodiment, GDNF ligand family proteins disclosed herein and heparin or heparan sulphate (and pharmaceutical compositions comprising same) can be used in a method for reducing the loss of pain sensitivity in a subject afflicted with a neuropathy. In one embodiment, the neuropathy is diabetic neuropathy. In some embodiments, the loss of pain sensitivity is a loss in thermal pain sensitivity. This methods include both prophylactic and therapeutic treatment.

In prophylactic treatment, a GDNF ligand family protein and heparin or heparan sulphate is administered to a subject at risk of developing loss of pain sensitivity (such a subject would be expected to be a subject with an early stage neuropathy). The treatment with a GDNF ligand family protein and heparin or heparan sulphate under such circumstances would serve to treat at-risk patients preventively.

In therapeutic treatment, a GDNF ligand family protein and heparin or heparan sulphate is administered to a subject who has experienced loss of pain sensitivity as a result of affliction with a neuropathy (such a subject would be expected to be a subject with a late stage neuropathy). The treatment with a GDNF ligand family protein and heparin or heparan sulphate under such circumstances would serve to rescue appropriate pain sensitivity in the subject.

### 4. Treatment of Viral Infections and Viral-Associated Neuropathies

Prophylactic treatment of infectious and viral neuropathies is contemplated. Prophylactic treatment is indicated after determination of viral infection and before onset of neuropathic pain. During treatment, a GDNF ligand family protein and heparin or heparan sulphate is administered to prevent appearance of neuropathic pain including but not limited to neuropathic pain associated with leprosy, Lyme disease, neuropathic pain associated with infection by a virus, particularly a virus selected from the group consisting of a herpes virus (and more particularly by a herpes zoster virus, which may lead to post-herpetic neuralgia), a human immunodeficiency virus (HIV), and a papilloma virus). In an alternative embodiment, a GDNF ligand family protein and heparin or heparan sulphate is administered to reduce the severity of neuropathic pain, should it appear.

Symptoms of acute viral infection often include the appearance of a rash. Other symptoms include, for example, the development of persistent pain in the affected area of the body, which is a common complication of a herpes zoster infection (shingles). Post-herpetic neuralgia can last for a month or more, and may appear several months after any rash-like symptoms have disappeared.

### 5. Treatment of Painful Diabetic Neuropathy

Prophylactic treatment of painful diabetic neuropathy is contemplated. Prophylactic treatment of diabetic neuropathies would commence after determination of the initial diagnosis of diabetes or diabetes-associated symptoms and before onset of neuropathic pain. Prophylactic treatment of painful diabetic neuropathy may also commence upon determining that a subject is at risk for developing diabetes or diabetes-associated symptoms. During treatment, a GDNF ligand family protein and heparin or heparan sulphate is administered to prevent appearance of neuropathic pain. In an alternative embodiment, a GDNF ligand family protein and heparin or heparan sulphate is administered to reduce the severity of neuropathic pain that has already appeared.

The following is an example of the practice of the invention. It is not to be construed as limiting the scope of the invention in any way.

### EXAMPLE

### Co-Administration of Heparin and Neublastin

Pre-cannulated (jugular vein) male Sprague Dawley rats were used. Neublastin was administered intravenously via a 1 cc syringe attached to the jugular catheter at a dose of 1 mg/kg either alone or with 16 kDa heparin in a composition containing (in PBS) 5 mM NaCitrate pH 7.0, 150 mM NaCl, and 0.01% Tween-80. The Neublastin polypeptide used in these experiments consisted of the carboxy terminal 113 amino acids of rat wild type Neublastin.

Blood samples were taken from the rats at various time points post dosing. Pharmacokinetics of the administered Neublastin at the various time points were measured by Ternary Complex ELISA.

TABLE 1
Concentration of Neublastin Detected in Serum after
Intravenous Administration of Neublastin Alone
or Co-Administration of Heparin and Neublastin

| Minutes after | Neublastin Alone | Neublastin + Heparin |
|---|---|---|
| Intravenous | (concentration in serum, | (concentration in serum, |
| Administration | ng/ml) | ng/ml) |
| 5 | 3,760 | 5,930 |
| 15 | 1,464 | 6,369 |
| 30 | 287 | 6,018 |
| 60 | 206 | 1,532 |
| 120 | 174 | not determined |

TABLE 2
Pharmacokinetic Parameters of Neublastin
after Intravenous Administration

| | | |
|---|---|---|
| Parameters after | | |
| Intravenous | | |
| Administration | Neublastin Alone | Neublastin + Heparin |

| | | | | |
|---|---|---|---|---|
| CL | 0.667 | L/(hr · kg) | 0.185 | L/(hr · kg) |
| T_{1/2} | 0.940 | hr | 1.77 | hr |
| MRT | 0.913 | hr | 0.58 | hr |
| V_{SS} | 0.609 | L/kg | 0.107 | L/kg |
| AUC | 1,500 | ng · hr/ml | 5,417 | ng · hr/ml |

As detailed in Tables 1 and 2, co-administration of heparin with Neublastin increased (as compared to administration of Neublastin alone) serum exposure of Neublastin, increased the area under the curve (AUC), decreased clearance of Neublastin, and increased the half life of the administered protein.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of increasing serum exposure of an administered glial cell line-derived neurotrophic factor (GDNF) ligand family protein, the method comprising administering to a subject via systemic delivery a pharmaceutical composition comprising (i) a GDNF ligand family protein, and (ii) an amount of heparin or heparan sulphate that increases serum exposure of the administered GDNF ligand family protein in the subject.

2. The method of claim 1, wherein the GDNF ligand family protein is a Neublastin polypeptide.

3. The method of claim 1, wherein the GDNF ligand family protein is a GDNF polypeptide.

4. The method of claim 1, wherein the GDNF ligand family protein is a Neurturin polypeptide.

5. The method of claim 1, wherein the GDNF ligand family protein is a Persephin polypeptide.

6. A method of treating a nervous system disorder, the method comprising administering to a subject that has a nervous system disorder, via systemic delivery, an effective amount of a pharmaceutical composition comprising (i) a GDNF ligand family protein, and (ii) an amount of heparin or heparan sulphate that increases serum exposure of the administered GDNF ligand family protein in the subject.

7. The method of claim 6, wherein the GDNF ligand family protein is a Neublastin polypeptide.

8. The method of claim 6, wherein the GDNF ligand family protein is a GDNF polypeptide.

9. The method of claim 6, wherein the GDNF ligand family protein is a Neurturin polypeptide.

10. The method of claim 6, wherein the GDNF ligand family protein is a Persephin polypeptide.

11. The method of claim 6, wherein the nervous system disorder is neuropathic pain or loss of pain sensitivity associated with a neuropathy.

12. The method of claim 1, wherein the GDNF ligand family protein is not conjugated to a polymer.

13. The method of claim 1, wherein the systemic delivery is intravenous administration.

14. The method of claim 1, wherein the systemic delivery is subcutaneous administration.


SEQUENCE LISTING (16 sequences)

SEQ ID NO: 1 - protein, 113 aa, Homo sapiens
AGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 2 - protein, 134 aa, Homo sapiens
SPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 3 - protein, 102 aa, homo sapiens
ARLGARPCGLRELEVRVSELGLGYASDETVLFRYCAGACEAAARVYDLGLRRLRQRRRLRRERVRAQPCCRPTAYEDEVSFLDAHSRYHTVHELSARECACV

SEQ ID NO: 4 - protein, 96 aa, Homo sapiens
ALSGPCQLWSLTLSVAELGLGYASEEKVIFRYCAGSCPRGARTQHGLALARLQGQGRAHGGPCCRPTRYTDVAFLDDRHRWQRLPQLSAAACGCGG

SEQ ID NO: 5 - protein, 220 aa, Homo sapiens
MELGLGGLSTLSHCPWPRRQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 6 - protein, 224 aa, mus musculus
MELGLAEPTALSHCLRPRWQSAWWPTLAVLALLSCVTEASLDPMSRSPAARDGPSPVLAPPTDHLPGGHTAHLCSERTLRPPPQSPQPAPPPPGPALQSPPAALRGARAARAGTRSSRARTTDARGCRLRSQLVPVSALGLGHSSDELIRFRFCSGSCRRARSQHDLSLASLLGAGALRSPPGSRPISQPCCRPTRYEAVSFMDVNSTWRTVDHLSATACGCLG

SEQ ID NO: 7 - protein, 224 aa, Rattus norvegicus
MELGLGEPTALSHCLRPRWQPALWPTLAALALLSSVTEASLDPMSRSPASRDVPSPVLAPPTDYLPGGHTAHLCSERTLRPPPQSPQPAPPPPGPALQSPPAALRGARAARAGTRSSRARATDARGCRLRSQLVPVSALGLGHSSDELIRFRFCSGSCRRARSPHDLSLASLLGAGALRSPPGSRPISQPCCRPTRYEAVSFMDVNSTWRTVDHLSATACGCLG

SEQ ID NO: 8 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 9 - protein, 240 aa, Mus musculus
MGFGPLGVNVQLGVYGDRIRGAAAGRDSKMKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 10 - protein, 211 aa, Rattus norvegicus
MKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCEAAETMYDKILKNLSRSRRLTSDKVGQACCRPVAFDDDLSFLDDSLVYHILRKHSAKRCGCI

SEQ ID NO: 11 - protein, 197 aa, Homo sapiens
MQRWKAAALASVLCSSVLSIWMCREGLLLSHRLGPALVPLHRLPRTLDARIARLAQYRALLQGAPDAMELRELTPWAGRPPGPRRRAGPRRRRARARLGARPCGLRELEVRVSELGLGYASDETVLFRYCAGACEAAARVYDLGLRRLRQRRRLRRERVRAQPCCRPTAYEDEVSFLDAHSRYHTVHELSARECACV

SEQ ID NO: 12 - protein, 195 aa, Mus muscukus
MRRWKAAALVSLICSSLLSVWMCQEGLLLGHRLGPALAPLRRPPRTLDARIARLAQYRALLQGAPDAVELRELSPWAARIPGPRRRAGPRRRRARPGARPCGLRELEVRVSELGLGYTSDETVLFRYCAGACEAAIRIYDLGLRRLRQRRRVRRERARAHPCCRPTAYEDEVSFLDVHSRYHTLQELSARECACV

SEQ ID NO: 13 - protein, 195 aa, Rattus norvegicus
MRCWKAAALVSLICSSLLSVWMCQEGLLLGHRLGPALAPLRRPPRTLDARIARLAQYRALLQGAPDAVELRELSPWVARPSGPRRRAGPRRRRARPGSRPCGLRELEVRVSELGLGYTSDETVLFRYCAGACEAAIRIYDLGLRRLRQRRRVRKERVRAHPCCRPTAYEDEVSFLDVHSRYHTLQELSARECACV

SEQ ID NO: 14 - protein, 156 aa, Homo sapiens
MAVGKFLLGSLLLLSLQLGQGWGPDARGVPVADGEFSSEQVAKAGGTWLGTHRPLARLRRALSGPCQLWSLTLSVAELGLGYASEEKVIFRYCAGSCPRGARTQHGLALARLQGQGRAHGGPCCRPTRYTDVAFLDDRHRWQRLPQLSAAACGCGG

SEQ ID NO: 15 - protein, 156 aa, Mus musculus
MAAGRLRILCLLLLSLHPSLGWVLDLQEASVADKLSFGKMAETRGTWTPHQGNNHVRLPRALAGSCRLWSLTLPVAELGLGYASEEKVIFRYCAGSCPQEARTQHSLVLARLRGRGRAHGRPCCQPTSYADVTFLDDQHHWQQLPQLSAAACGCGG

SEQ ID NO: 16 - protein, 156 aa, Rattus norvegicus
MAAGRLRILFLLLLSLHLGLGWVLDLQEAPAADELSSGKMAETGRTWKPHQGNNNVRLPRALPGLCRLWSLTLPVAELGLGYASEEKIIFRYCAGSCPQEVRTQHSLVLARLRGQGRAHGRPCCQPTSYADVTFLDDHHHWQQLPQLSAAACGCGG